(19) United States
(12) Patent Application Publication
(10) Pub. No.: US 2010/0305318 A1
(43) Pub. Date: Dec. 2, 2010

(54) Process for the Manufacture of Anagrelide Hydrochloride Monohydrate

(75) Inventors: Manjunath Narayan Bhanu, Pune (IN); Samir Naik, Thane (IN); Sadanand Maurya, Dombivli (East) (IN); Kishor Mahajan, Dombivli (West) (IN)

Correspondence Address: Florek & Endres PLLC, 1156 Avenue of the Americas, Suite 600, New York, NY, 10036, US

(73) Assignee: WATSON PHARMA PRIVATE LIMITED, Andheri (East), Mumbai (IN)

(21) Appl. No.: 12/808,365

(22) PCT Filed: Dec. 18, 2008
(86) PCT No.: PCT/IN2008/000844

§ 371 Date: Jun. 15, 2010

(30) Foreign Application Priority Data
Dec. 19, 2007 (IN) 2498/MUM/2007

(51) Int. Cl. C07D 487/04

(52) U.S. Cl. 544/250

(57) ABSTRACT

The present invention relates to a process for preparation of Anagrelide Hydrochloride Monohydrate.

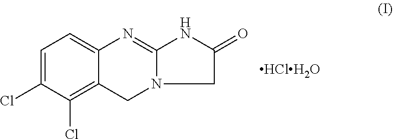

### FIELD OF THE INVENTION

The present invention relates to an improved process for preparation of the Hydrochloride Monohydrate salt of 6,7-dichloro-1,5-dihydroimidazo (2,1-b) quinazolin-2(3H)-one, commonly known as Anagrelide Hydrochloride Monohydrate, a known potent blood platelet reducing agent.

### BACKGROUND OF THE INVENTION

Anagrelide (6,7-dichloro-1,5-dihydroimidazo[2,1-1)]quinazolin-2(3H)-one Hydrochloride Monohydrate (formula I) is a potent blood platelet reducing agent. A number of U.S. Patents have issued on Anagrelide and its method of making including U.S. Pat. Nos. 3,932,407; 4,146,718; 4,208,521; 4,357,330;RE31,617; and 5,801,245. These patents are incorporated herein by reference.

Commercially, as discussed in U.S. Pat. No. 5,801,245, Anagrelide has been prepared as the Hydrochloride Monohydrate (formula I) from the intermediate, ethyl N-(6-amino-2,3-dichlorobenzyl)glycine by reaction with cyanogen bromide in hot alcohol solution, or, preferentially, by reaction with cyanogen bromide in an aprotic solvent to give an iminoquinazoline intermediate which is isolated and then reacted with a base in a hot solution of alcohol to form Anagrelide base.

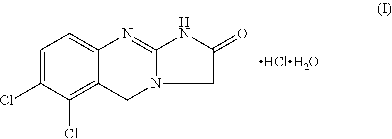

The Hydrochloride Monohydrate Anagrelide salt is prepared by adding hydrochloric acid to a methanol slurry of Anagrelide base and heating to reflux. The Hydrochloride salt is then hydrated in a high humidity chamber. These two steps are time-consuming and the yield of Hydrochloride salt can be poor due to competing acid hydrolysis of the lactam ring and methyl ester formation. After 15 minutes at reflux, the isolated yield is 62%, and this decreases to 40% after 2 hours.

Normally, salts are prepared when the free base has undesirable properties such as poor solubility or a non-solid physical state. In this case, both Anagrelide base and the Hydrochloride salt are solids with low aqueous solubility. In addition, the water of crystallization can accelerate decomposition of the parent molecule through hydrolysis of the lactam ring presenting long-term stability problems for pharmaceutical Anagrelide formulations.

U.S. Pat. No. 5,391,737 discloses a process for manufacturing Anagrelide Hydrochloride semihydrate.

Hence there is a need for a simple and efficient process to manufacture the Hydrochloride Monohydrate salt on a commercial scale. The present invention thus seeks to provide a simple and efficient process for commercial scale manufacture of the Hydrochloride Monohydrate salt of Anagrelide.

### THE OBJECT OF THE INVENTION

The object of the present invention is a process for preparation of the Hydrochloride Monohydrate salt of Anagrelide using a user friendly process, which may be commercially feasible, and results in an improved yield and quality of the final Anagrelide Hydrochloride Monohydrate.

### SUMMARY OF THE INVENTION

The present invention relates to a process for preparing Anagrelide Hydrochloride Monohydrate (formula I). Preferably, the process can be used on a commercial scale.

The process according to the present invention comprises:

a) preparing a suspension of Anagrelide base in an alcohol, preferably a C₁-C₅ aliphatic alcohol;
b) heating the suspension, preferably under reflux conditions;
c) adding about 0.1 to about 4 volumes of water, preferably about 0.15 to about 2 volumes of water and more preferably about 0.2 to about 1 volume of water to the suspension of step (b);
d) acidifying the suspension with hydrochloric acid;
e) refluxing the acidified reaction mixture;
f) cooling the refluxed reaction mixture;
g) collecting the Anagrelide Hydrochloride Monohydrate crystals from the cooled reaction mixture; and
h) drying the collected Anagrelide Hydrochloride Monohydrate crystals.

### DETAILED DESCRIPTION OF THE INVENTION

One embodiment of the present invention for preparing Anagrelide Hydrochloride Monohydrate crystals comprises:

a) preparing a suspension of Anagrelide base in an alcohol, preferably a C₁-C₅ aliphatic alcohol;
b) heating the suspension, preferably under reflux conditions;
c) adding about 0.1 to about 1 w/v of water based upon the weight of the Anagrelide base, preferably about 0.15 to about 0.5 w/v of water based upon the weight of the Anagrelide base and more preferably about 0.2 to about 0.4 w/v of water based upon the weight of the Anagrelide base to the suspension;
d) acidifying the suspension with hydrochloric acid;
e) refluxing the acidified reaction mixture for about 1 to about 20 minutes, preferably about 2 to about 15 minutes and most preferably about 3 to about 10 minutes;
f) rapidly cooling the refluxed reaction mixture to a temperature of about 0° C. to about 25° C., preferably about 5° C. to about 20° C. and most preferably about 10° C. to about 15° C.;
g) collecting the Anagrelide Hydrochloride Monohydrate crystals from the cooled reaction mixture; and
h) drying the collected Anagrelide Hydrochloride Monohydrate crystals until a water content of about 5-7% is obtained.

Although Anagrelide base for use in the present invention can be prepared by any method known in the art, the following reaction sequence (Scheme I) is one possible method for preparation of the Anagrelide base for use in the present invention.

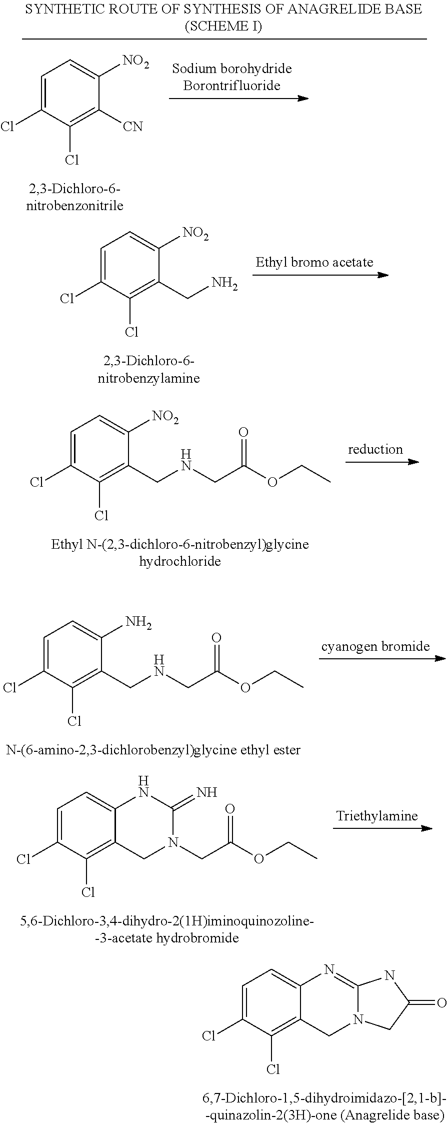

The Anagrelide base prepared according to Scheme I or any other conventional process known in the art is then converted to the Hydrochloride Monohydrate salt within the scope of this invention as described above.

In an alternate embodiment of the present invention, the Anagrelide base is converted into Anagrelide Hydrochloride Monohydrate by the following process:

a) suspending the Anagrelide base in a lower alcohol selected from the group consisting of methanol, ethanol propanol, isopropanol or mixtures of the foregoing;
b) heating the suspension of step (a) to about 60° C. to about 90° C., preferably about 65° C. to about 85° C., most preferably about 70° C. to about 80° C.;
c) adding about 0.25 to about 4 volumes of water to the reaction mixture of step (b);
d) acidifying the reaction mixture of step (c) by adding about 0.8 w/v to about 1.2 w/v of concentrated HCl based upon the weight of the Anagrelide base, preferably about 1 w/v of concentrated HCl (33%-36.5% aqueous solution of HCl), to the reaction mixture from step (c);
e) refluxing the acidified reaction mixture for 3 to 10 minutes;
f) cooling the refluxed reaction mixture rapidly to about 0° C. to about 25° C., preferably about 5° C. to about 20° C. and most preferably about 10° C. to about 15° C.;
g) collecting the Anagrelide Hydrochloride Monohydrate crystals from the cooled reaction mixture; and
h) drying the Anagrelide Hydrochloride Monohydrate crystals until a water content between 5 -7% is obtained.

In another embodiment of the present invention, the heating of the alcohol suspension of the Anagrelide base is conducted under reflux conditions by first heating the alcohol, preferably methanol or ethanol, then adding the Anagrelide base to the hot alcohol and stirring. The reflux of the alcohol suspension continues for about 5-20 minutes, preferably about 10-15 minutes before about 0.8 w/v to about 1.2 w/v of water based upon the weight of the Anagrelide base is added to alcohol suspension. When the water is added to the alcohol suspension, the temperature of the alcohol suspension should be maintained. About 2-10 minutes after the water is added, hydrochloric acid is added to the reaction mixture.

The hydrochloric acid may be added dropwise in a concentrated form, via hydrochloride gas bubbling techniques or by a dropwise addition of a diluted form of hydrochloric acid. If the hydrochloric acid is added in a diluted form, a reduction in the amount of water added to the alcohol suspension in step (c) may be required. In one embodiment of the present invention, the hydrochloric acid is added in a concentrated form over a period of about 1 to about 5 minutes, preferably about 2 to about 4 minutes and most preferably about 3 minutes.

After the required amount of hydrochloric acid is added to the reaction mixture, the reaction mixture is heated, preferably refluxed, for about 2-10 minutes. The reaction mixture is rapidly cooled to about 0° C. to about 25° C., preferably about 5° C. to about 20° C. and most preferably about 10° C. to about 15° C. using conventional cooling techniques such as an ice bath, and stirred for approximately 10-20 minutes.

The solid materials are collected, preferably by filtration, and washed with an appropriate material such as a lower aliphatic alcohol such as methanol, ethanol or IPA. The washed solid material is vacuum dried at about 25-35° C. for about 4-6 hours or until the moisture content of the Anagrelide Hydrochloride Monohydrate crystals is about 5 to about 7%. The moisture content can be determined by conventional measurement techniques.

In one embodiment of the present invention the vacuum pressure for the drying of the Anagrelide Hydrochloride Monohydrate crystals can range from about 500 mm of Hg to about 760 mm of Hg, preferably about 600 mm of Hg to about 750 mm of Hg and most preferably about 650 mm of Hg to about 750 mm of Hg.

The Anagrelide Hydrochloride Monohydrate prepared in accordance with the present invention may be mixed with at least one additional conventional pharmaceutical excipient to prepare a pharmaceutical dosage form such as a tablet, capsule or solution.

The following examples are illustrative and are not meant to limit the scope of the claimed invention.

### EXAMPLE 1

### Preparation of 2,3-dichloro-6-nitrobenzylamine

2,3-dichloro-6-nitrobenzonitrile, 500 gm, was dissolved in 7500 ml of THF in a clean dry RB flask maintaining a nitrogen blanket. 139.3 am of sodium borohydride was added at 25-30° C. The suspension was cooled to 15-20° C. 653 gms of Borontrifluoride etherate was added to the reaction mixture over a period of about 2 hours while maintaining the temperature between 15-30° C. After the addition was complete, the temperature was raised about 65° C. and maintained at reflux for 2 hours. The reaction mixture was cooled to about 0-5° C. A mixture of 2.0 liter water and 200 ml concentrated HCl was slowly added to the reaction mixture while maintaining the temperature below 20° C. After the addition was complete, THF was distilled from the reaction mixture under vacuum maintaining temperature below 50° C. The reaction mixture was cooled to about 10-15° C. and 2.0 liter ethylacetate was added. The pH of reaction mixture was adjusted to 10-11 using 25% aqueous sodium hydroxide solution while maintaining the temperature below 15° C. The ethylacetate layer was separated and concentrated to about half the original volume. The reaction mixture was cooled to 15-20° C. and 500 ml IPA-HC1 was added slowly, maintaining temperature below 30° C. After cooling to about 0-5° C., the solids obtained were filtered and washed with 500 ml chilled ethylacetate. The solids were dried to obtain 2,3-dichloro-6-nitrobenzyl amine hydrochloride (450 gms),

### EXAMPLE 2

### Preparation of Ethyl N-(2,3-dichloro-6-nitrobenzyl)glycine Hydrochloride

To a suspension of 450 gms of 2,3-dichloro-6-nitrobenzylamine in 2250 ml of 1,4-dioxane was added 442 gms of triethylamine slowly under stirring at 25-35° C. The reaction mixture was heated to 90-95° C. and 583.6 gms of ethyl bromoacetate in 900 ml of 1,4-dioxane was added slowly to the reaction mixture over a period of 2 hours maintaining the temperature at 90-100° C. After the addition was complete the reaction mixture was stirred at 95-100° C. for about 2 hours. 1,4-dioxane was completely distilled off under vacuum while maintaining the temperature below 60° C. The reaction mixture was cooled to 25° C., and 1800 ml water and 1800 ml ethylacetate were added to the reaction mixture. The pH of the reaction mixture was adjusted to 10-11 using 25% sodium hydroxide solution. The layers were separated, and the organic layer of ethyl acetate was concentrated under vacuum about 4 volumes. The reaction mixture was contacted with 500 ml of IPA at a temperature of about 20-30° C. HCl solution was added slowly at 20-30° C. under stirring. Another 1800 ml ethylacetate was added and cooled to 0-5° C. and stirred for about 1 hour. The solids obtained were filtered and washed with chilled ethyl acetate. After drying at 50° C., ethyl N-(2,3-dichloro-6-nitrobenzyl)glycine hydrochloride (380 gms) was obtained.

### EXAMPLE 3

### Preparation of N-(6-amino-2,3-dichlorobenzyl)glycine Ethyl Ester

2660 ml of concentrated HCl was added to 380 gms of ethyl N-(2,3-dichloro-6-nitrobenzyl) glycine hydrochloride at 20-30° C. and stirred for 15 minutes. The reaction mixture was cooled to 10° C., and a solution of 1621.5 gm of stannous chloride dihydrate in 4180 ml concentrated hydrochloric acid was added slowly over 90 minutes while maintaining the temperature between 10-15° C. After the addition was completed, the temperature was raised and the reaction mixture was stirred at 25° C. for 1 hour. The reaction mixture was cooled to 10-15° C., and solids obtained were filtered and washed with 150 ml chilled concentrated hydrochloric acid. The solids were suspended in 1.5 liter water and chilled to 5° C. The pH of the reaction mixture was adjusted to 10-11 using 700 ml liquor ammonia solution while maintaining the temperature below 5° C. The solids obtained were filtered and extracted with 600 ml isopropyl ether thrice. The filtrate was extracted twice with 300 ml isopropyl ether. All the isopropyl ether extracts were collected together and washed with 600 ml brine solution. The reaction mixture was concentrated under vacuum while maintaining the temperature below 40° C. to obtain a thick slurry. 560 ml of hexane was added to the slurry and about 500 ml hexane was distilled out under vacuum while maintaining the temperature below 40° C. Further, 560 ml hexane was added, slowly heated to 60° C. and stirred for 15 minutes. The reaction mixture was gradually cooled to 25° C. and stirred for 2 hours. The product was filtered and washed with 200 ml hexane. After drying under vacuum at 35° C., N-(6-amino-2,3-dichlorobenzyl)glycine ethyl ester (209 gm) was obtained.

### EXAMPLE 4

### Preparation of 5,6-dichloro-3,4-dihydro-2(1H)iminoquinozoline-3-acetate Hydrobromide

255 gm of N-(6-amino-2,3-dichlorobenzyl)glycine ethyl ester was dissolved in 1785 ml toluene and cooled to 5-10° C. A solution of 146.2 gms of cyanogen bromide in 1785 ml toluene was added to the reaction mixture in about 1 hour while maintaining the temperature below 20° C. After the addition was complete, the reaction mixture was heated to reflux for 4 hours. The reaction mixture was cooled to 30° C., and the solids were filtered and slurried in 1000 ml toluene at 25-35° C. The solids were filtered and washed with 1000 ml toluene twice.

The crude product (325 gm) was obtained after drying under vacuum at 40° C. for 3 hours.

### PURIFICATION

325 gm of crude 5,6-dichloro-3,4-dihydro-2(1H)iminoquinozoline-3-acetate hydrobromide was suspended in 9.75 liter ethanol and heated to reflux for 30 minutes, resulting in a clear solution. 7 gm of activated charcoal was added, and the reaction mixture was heated to reflux and maintained for 30 minutes. The reaction mixture was filtered hot over a hyflo bed. The hyflo bed was washed with 300 ml hot ethanol. The clear filtrate was cooled gradually to about 30° C. and stirred for 1 hour. The pure product obtained was filtered and washed with 100 ml ethanol. 5,6-dichloro-3,4-dihydro2(1H)iminoquinozoline-3-acetate hydrobromide (220 gm) was obtained after drying at 40° C. under vacuum.

### EXAMPLE 5

### Preparation of 6,7-dichloro-1,5-dihydroimidazo-[2,1-b]quinazolin-2(3H)-one (Anagrelide base)

197 gm of 5,6-dichloro-3,4-dihydro-2(1H)iminoquinozoline-3-acetate hydrobromide was suspended in 3152 ml ethanol under stirring at 30° C. 83.2 gm of triethylamine was added to the reaction mixture. The reaction mixture was heated to reflux and maintained 4 hours. The reaction mixture was further cooled to 25° C., the solids obtained were filtered and washed with 3 liters of distilled water and then with 300 ml ethanol. The wet solids were suspended in a mixture of 1507.5 ml ethanol and 167.5 ml water and stirred at 30° C. for 15 minutes. The reaction mixture was heated to reflux at about 80° C. and maintained 4 hours. The solids were filtered hot at 65-70° C. and washed with 300 ml ethanol and suck dried for 1 hour. The wet product was dried at 25-35° C. under vacuum for 6 hours to get Anagrelide Base (125 gm)

### EXAMPLE 6

### Preparation of Anagrelide Hydrochloride Monohydrate

3750 ml ethanol was heated to about 75° C. in a clean dry round bottom flask. 125 gm of Anagrelide base was added to the hot ethanol under stirring. The reaction mixture was refluxed at 78-80° C. for 10 minutes. 37.5 ml of distilled water was added to the reaction mixture and stirred at 78-80° C. for 5 minutes. 125 ml concentrated hydrochloric acid was added to the reaction mixture in about 3 minutes while maintaining the reflux temperature. After the addition was complete, the reaction mixture was maintained at 78-80° C. for about 5 minutes. The reaction mixture was cooled to 10-15° C. and stirred for 15 minutes. The solids obtained were filtered and washed with 250 ml ethanol. The product was dried at 30° C. under vacuum for about 4-6 hours until water content of the product was between 5.2% to 6.5% to get 125 gm of Anagrelide Hydrochloride Monohydrate.

The present invention has been particularly described in conjunction with a specific preferred embodiment. It should be evident that many alternatives, modifications and variations will be apparent to those skilled in the art in light of the foregoing description. It is therefore contemplated that the appended claims will embrace any such alternatives, modifications and variations as falling within the true scope and spirit of the present invention.

We claim:

1. A process for preparation of Anagrelide Hydrochloride Monohydrate, which comprises;
a) preparing a suspension of Anagrelide base in an alcohol;
b) heating the suspension;
c) adding water to the suspension;
d) acidifying the suspension;
e) refluxing the suspension;
f) cooling the suspension;
g) filtering the cooled suspension to collect the Anagrelide Hydrochloride Monohydrate crystals; and
h) drying the wet Anagrelide Hydrochloride Monohydrate crystals.

2. The process for preparation of Anagrelide Hydrochloride Monohydrate as claimed in claim 1 wherein the alcohol of step (a) is methanol or ethanol.

3. The process for preparation of Anagrelide Hydrochloride Monohydrate as claimed in claim 1 wherein the Anagrelide base suspension of step (b) is refluxed.

4. The process for preparation of Anagrelide Hydrochloride Monohydrate as claimed in claim 1 wherein the water that is added to the suspension in step (c) is about 0.2 w/v to about 0.4 w/v based upon the weight of the Anagrelide base.

5. The process for preparation of Anagrelide Hydrochloride Monohydrate as claimed in claim 1 wherein concentrate HCl is added in the amount of about 0.8 w/v to about 1.2 w/v based upon the weight of the Anagrelide base.

6. The process for preparation of Anagrelide Hydrochloride Monohydrate as claimed in claim 1 wherein the suspension is cooled to about 10° C. to about 15° C.

7. The process for preparation of Anagrelide Hydrochloride Monohydrate as claimed in claim 1 wherein Anagrelide Hydrochloride Monohydrate crystals are dried at about 20° C. to about 40° C.

8. The process for preparation of Anagrelide Hydrochloride Monohydrate as claimed in claim 1 wherein the Anagrelide Hydrochloride Monohydrate crystals are dried under vacuum at a pressure of about 600 mm of Hg to about 750 mm of Hg.

9. The process for preparation of Anagrelide Hydrochloride Monohydrate as claimed in claim 1 wherein the moisture content of the dried crystals is about 5.0% to about 7.0%.
